(19) United States
(12) Patent Application Publication
(10) Pub. No.: US 2024/0390649 A1
(43) Pub. Date: Nov. 28, 2024

(54) SECUREMENT DEVICE, KIT, AND METHOD OF USING SECUREMENT DEVICE

(71) Applicant: 3M INNOVATIVE PROPERTIES COMPANY, St. Paul, MN (US)

(72) Inventor: Ganesh P. D. Kannan, Bangalore (IN)

(21) Appl. No.: 18/292,202

(22) PCT Filed: Jul. 27, 2022
(86) PCT No.: PCT/IB2022/056934

§ 371 Date: Jan. 25, 2024

Related U.S. Application Data
(60) Provisional application No. 63/229,729, filed on Aug. 5, 2021.

(51) Int. Cl. A61M 25/02

(52) U.S. Cl. CPC A61M 25/02; A61M 2025/0246; A61M 2025/0273

(57) ABSTRACT

A securement device (100) for a catheter assembly (10) includes a base (102) having a first major surface (104) and an opposing second major surface (106). The base (102) defines a through cavity (108). The base (102) is detachably secured to a skin (12) of a user, such that an injection site (22) associated with the catheter assembly is disposed within the through cavity (108). The through cavity (108) receives at least a portion of the catheter assembly (10) therein. The securement device (100) also includes a casing (116) that encloses the through cavity (108) and the catheter assembly (100). The casing (116) engages with the catheter assembly (100) to secure the catheter assembly (100) within the through cavity (108). The casing (116) includes a material having a self-healing property. The casing (116) at least partially ruptures to receive at least one lumen (18) of the catheter assembly (100) therethrough. Moreover, upon receiving the at least one lumen (18) of the catheter assembly (100) therethrough, the casing (116) forms a seal (140) around the at least one lumen (18).

### TECHNICAL FIELD

The present disclosure relates to a securement device for securing a catheter assembly, a kit including the securement device, and a method of using the securement device.

### BACKGROUND

Catheter assemblies may be used for various purposes, such as feeding, air supply, and/or liquid removal. In some cases, catheter assemblies may be used to administer medications and fluids to a user. In many instances, catheter assemblies may need to be secured to a skin of the user to prevent movement of the catheter assemblies. For example, the catheter assemblies may need to be secured to the skin of the user to prevent the catheter assemblies from being pulled out, or otherwise moved in ways that may adversely affect functioning of the catheter assemblies.

The catheter assemblies are generally secured to the skin of the user using conventional catheter securement units, such as tapes, patches, and sutures. However, such conventional catheter securement units may not properly secure the catheter assemblies to the skin. Specifically, conventional catheter securement units may not properly stabilize the catheter assemblies, which may cause undesirable movement of the catheter assemblies during use. In some cases, use of conventional catheter securement units may result in various complications, such as dislodgment and accidental removal of the catheter assemblies from the skin of the user, phlebitis, extravasation/infiltration, leakage, occlusion, and bloodstream infections. Additionally, conventional methods to secure the injection site may create discomfort for the patients due to high fluid pressure in the catheter assembly.

Catheter securement units such as tapes or patches may allow partial or micro movement of the catheter assembly which can lead to discomfort for the patients. Further, conventional catheter securement units may not provide proper coverage of an injection site, which may result in infection at the injection site. Moreover, use of sutures to secure the catheter assemblies to the skin may lead to suture related infections and complications. Thus, an improved securement device is needed to overcome the abovementioned challenges of conventional securement devices and methods.

### SUMMARY

Generally, the present disclosure relates to a securement device for use with a catheter assembly. The present disclosure further relates to a kit including the catheter assembly and the securement device, and a method of using the securement device.

In a first aspect, the present disclosure provides a securement device for securing a catheter assembly. The securement device includes a base having a first major surface and an opposing second major surface. The base defines a through cavity extending from the first major surface to the second major surface. The base is configured to be detachably secured to a skin of a user, such that an injection site associated with the catheter assembly is disposed within the through cavity. The through cavity receives at least a portion of the catheter assembly therein. The securement device also includes a casing mounted on the first major surface of the base and enclosing the through cavity and the catheter assembly. The casing is configured to engage with the catheter assembly to secure the catheter assembly within the through cavity. The casing includes a material having a self-healing property. The casing at least partially ruptures to receive at least one lumen of the catheter assembly therethrough. Moreover, upon receiving the at least one lumen of the catheter assembly therethrough, the casing forms a seal around the at least one lumen.

In a second aspect, the present disclosure provides a kit. The kit includes a catheter assembly. The catheter assembly includes a catheter hub, at least one lumen at least partially received within the catheter hub, and a cannula at least partially received within the catheter hub. The cannula is configured to be at least partially inserted into a skin of a user at an injection site. The kit also includes a securement device. The securement device includes a base having a first major surface and an opposing second major surface. The base defines a through cavity extending from the first major surface to the second major surface. The base is configured to be detachably secured to the skin of the user, such that an injection site associated with the catheter assembly is disposed within the through cavity. The through cavity receives at least a portion of the catheter assembly therein. The securement device also includes a casing mounted on the first major surface of the base and enclosing the through cavity and the catheter assembly. The casing is configured to engage with the catheter assembly to secure the catheter assembly within the through cavity. The casing includes a material having a self-healing property. The casing at least partially ruptures to receive at least one lumen of the catheter assembly therethrough. Moreover, upon receiving the at least one lumen of the catheter assembly therethrough, the casing forms a seal around the at least one lumen.

In a third aspect, the present disclosure provides a method of using a securement device including a base and a casing mounted on a first major surface of the base. The method includes detachably securing the base to a skin of a user, such that a second major surface of the base opposite to the first major surface faces the skin. The catheter assembly is at least partially received within a through cavity of the base and enclosed by the casing. The method also includes engaging at least one lumen of the catheter assembly with the casing, such that the casing at least partially ruptures and receives the at least one lumen therethrough. The casing includes a self-healing material, such that upon receiving the at least one lumen therethrough, the casing forms a seal around the at least one lumen. The method further includes engaging the catheter assembly with the casing to secure the catheter assembly within the casing.

### BRIEF DESCRIPTION OF THE DRAWINGS

Exemplary embodiments disclosed herein may be more completely understood in consideration of the following detailed description in connection with the following figures. The figures are not necessarily drawn to scale. Like numbers used in the figures refer to like components. However, it will be understood that the use of a number to refer to a component in a given figure is not intended to limit the component in another figure labeled with the same number.

FIG. 1 illustrates a schematic perspective view of a catheter assembly according to an embodiment of the present disclosure;

FIG. 2 illustrates a schematic partially exploded perspective view of a securement device for securing the catheter assembly of FIG. 1 according to an embodiment of the present disclosure;

FIG. 3 illustrates a schematic cross-sectional view of a base of the securement device of FIG. 2 according to an embodiment of the present disclosure;

FIG. 4A illustrates a schematic perspective view of the securement device and the catheter assembly of FIG. 2 according to an embodiment of the present disclosure;

FIG. 4B illustrates a schematic perspective view of the securement device of FIG. 2 and the catheter assembly according to an embodiment of the present disclosure;

FIG. 5 illustrates a schematic cross-sectional view of the securement device shown in FIG. 4B along a plane A-A1 according to an embodiment of the present disclosure;

FIG. 6 illustrates a schematic top view of the securement device of FIG. 2 and the catheter assembly according to an embodiment of the present disclosure;

FIG. 7 illustrates a schematic cross-sectional view of the securement device shown in FIG. 6 along a plane B-B1 according to an embodiment of the present disclosure;

FIG. 8 illustrates a schematic block diagram of a kit according to an embodiment of the present disclosure;

FIG. 9 is a flowchart for a method of using the securement device according to an embodiment of the present disclosure; and

FIGS. 10A to 10F illustrate schematic perspective views of various steps for using the securement device according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” and variations thereof, are used broadly and encompass both direct and indirect attachments, connections, and couplings.

As used herein, the terms “layer,” “sheet,” and “dressing,” or variations thereof, are used to describe an article having a thickness that is small relative to its length and width.

As used herein, the term “polymer” refers to both materials prepared from one monomer such as a homopolymer or to materials prepared from two or more monomers such as a copolymer, terpolymer, or the like. Likewise, the term “polymerize” refers to the process of making a polymeric material that can be a homopolymer, copolymer, terpolymer, or the like. The term “copolymer” refers to a polymeric material prepared from at least two different monomers.

As used herein, the term “modulus of elasticity” (also interchangeably referred to as Young's modulus or storage modulus) refers to a quantity that measures a material's resistance to being deformed elastically when a stress is applied to it. Essentially, the modulus of elasticity is a quantitative measure of the stiffness of an elastic material that measures the ability of the material under test to recover to its original shape or size. Modulus of elasticity can be calculated using an equation derived by Hooke's law, where the modulus of elasticity is equal to a ratio of stress to strain (i.e., ratio of applied force to change in fraction of size).

The present disclosure relates to a securement device for use with a catheter assembly, a kit including the securement device, and a method of using the securement device. The securement device includes a base having a first major surface and an opposing second major surface. The base defines a through cavity extending from the first major surface to the second major surface. The base is configured to be detachably secured to a skin of a user, such that an injection site associated with the catheter assembly is disposed within the through cavity. The through cavity receives at least a portion of the catheter assembly therein. The securement device also includes a casing mounted on the first major surface of the base and enclosing the through cavity and the catheter assembly. The casing is configured to engage with the catheter assembly to secure the catheter assembly within the through cavity. The casing includes a material having a self-healing property. The casing at least partially ruptures to receive at least one lumen of the catheter assembly therethrough. Moreover, upon receiving the at least one lumen of the catheter assembly therethrough, the casing forms a seal around the at least one lumen.

Conventional catheter securement units, such as tapes, patches, and sutures may not properly secure the catheter assemblies to the skin of the user. Specifically, the conventional catheter securement units may not properly stabilize the catheter assemblies, thereby allowing undesirable movement of the catheter assemblies during use. In some cases, use of conventional catheter securement units may result in various complications, such as dislodgment and accidental removal of the catheter assemblies from the skin of the user, phlebitis, extravasation, occlusion/infiltration, leakage, and bloodstream infections. Furthermore, conventional catheter securement units may not provide proper coverage of an injection site, which may result in infection at the injection site. Moreover, use of sutures to secure the catheter assemblies to the skin may lead to suture related infections.

The securement device secures the catheter assembly firmly onto the skin of the user. Specifically, the securement device may firmly secure various components (such as a catheter hub, one or more lumens, and a cannula) of the catheter assembly to the skin of the user. The securement device may be manufactured as an integral part of the catheter assembly, or it may be retrofitted on existing catheter assemblies. The securement device prevents movement of the catheter assembly and eliminates requirements of any additional tapes, patches, or sutures. It should be noted that once the securement device is placed onto the injection site, no additional dressing may be required. Further, the casing includes a self-healing material. The self-healing material of the casing of the securement device forms a seal around a needle or one or more lumens. Further, the securement device includes one or more guide pins that engage with the lumens to inhibit the movement of the lumens relative to the casing. Moreover, one or more stabilization lugs of the securement device engages with the catheter hub of the catheter assembly to inhibit the movement of the catheter hub relative to the casing. Additionally, a transparent or translucent material of the casing allows the user to observe the injection site when the securement device is secured onto the skin.

Further, the base of the securement device may be removably secured to the skin of the user. The base includes one or more pull tabs that may be gripped by user and pulled to detach the base from the skin. An adhesive layer firmly secures the base on the skin of the user. The adhesive layer includes a stretch release adhesive such that based on pulling of the base, the adhesive layer loses its adhesion property. Subsequently, the base can be released easily to remove the securement device from the skin of the user. In other words, the base may be easily removed from the skin of the user without causing discomfort to the user. Thus, the securement device may prevent dislodgement and accidental removal of the catheter assembly from the skin of the user. Further, an anti-microbial gel may be injected into the casing. An opening may be formed in the casing due to insertion of a needle that injects the anti-microbial gel. After removal of the needle, the opening may reseal due to the self-healing material of the casing. Thus, the casing may remain intact after injection of the anti-microbial gel. The anti-microbial gel surrounds and protects the injection site from infections and keeps the injection site sterile. Further, the anti-microbial gel may also nourish the skin of the user and may keep the skin moist. Thus, the securement device may prevent various complications, such as phlebitis, extravasation/infiltration, leakage, occlusion, and bloodstream infections that may otherwise occur due to movement of the catheter assembly.

Thus, the securement device of the present disclosure may firmly secure the catheter assembly to the skin of the user, may inhibit undesirable movement of the catheter assembly, may prevent infection at the injection site, and may facilitate easy removal of the securement device after use.

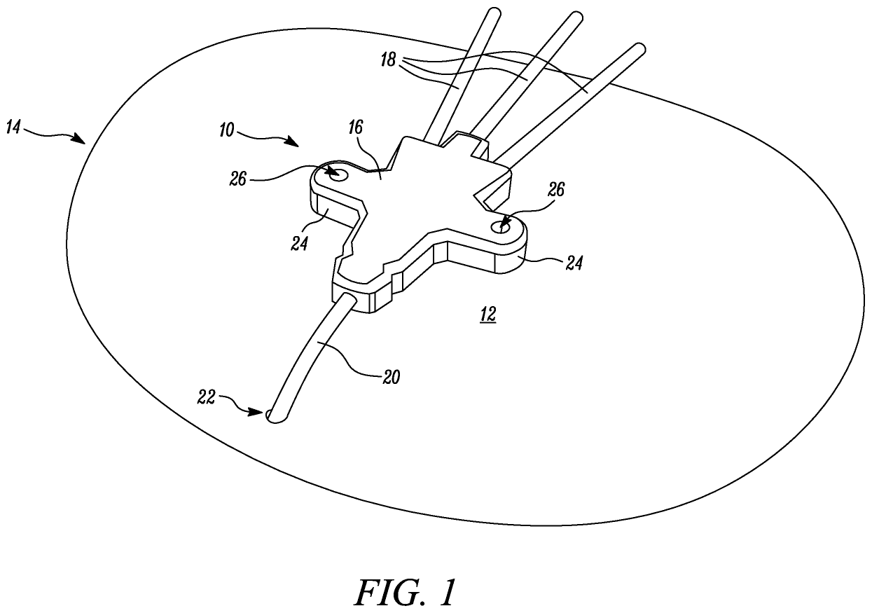

Referring now to Figures, FIG. 1 illustrates a schematic perspective view of a catheter assembly 10 according to an embodiment of the present disclosure. In FIG. 1, the catheter assembly 10 is partially disposed on a skin 12 of a user 14. In some cases, the user 14 may be a patient undergoing an intravenous therapy. As illustrated in FIG. 1, the catheter assembly 10 includes a catheter hub 16, at least one lumen 18 at least partially received within the catheter hub 16, and a cannula 20 at least partially received within the catheter hub 16.

In the illustrated embodiment of FIG. 1, the one or lumens 18 include three lumens 18. In other words, the catheter assembly 10 is a triple lumen catheter assembly. However, a number of the lumens 18 may vary based on desired application and/or infusion attributes. The one or more lumens 18 are fluidly connected with the cannula 20. In the illustrated embodiment of FIG. 1, the cannula 20 is configured to be at least partially inserted into the skin 12 of the user 14 at an injection site 22.

In the illustrated embodiment of FIG. 1, the catheter hub 16 includes a pair of opposing wings 24 and one or more holes 26. Specifically, in the illustrated embodiment of FIG. 1, the catheter hub 16 includes a corresponding hole 26 on each of the pair of opposing wings 24. The holes 26 are embodied as suture holes. In some situations, the holes 26 may optionally allow suturing of the catheter assembly 10 with the skin 12 of the user 14.

In some embodiments, the catheter assembly 10 may include a central venous catheter (CVC). The CVC may be referred to as a central line, a central venous line, or a central venous access catheter. The CVC may be used to access large, centrally located veins (not shown), which is often required for critically ill patients, for patients requiring prolonged intravenous therapies for reliable vascular access, and to administer fluids that may harm smaller peripheral veins. Therefore, in some embodiments, at least a portion of the cannula 20 may be inserted into veins located at a neck (e.g., an internal jugular vein), into veins located at a chest (e.g., a subclavian vein or axillary vein), or into veins located at a groin (e.g., a femoral vein) of the user 14. In some embodiments, the catheter assembly 10 may include a peripherally inserted central catheter (PICC). The PICC may be suitable for insertion into veins located at an arm of the user 14. However, the catheter assembly 10 may include any suitable type of catheter, which may be selected based on desired application attributes.

Thus, the catheter assembly 10 may be used to administer an intravenous therapy (e.g., medication or fluids for parenteral nutrition), to obtain blood for analysis, and/or to provide an access point for blood-based treatments, such as dialysis or apheresis. The catheter assembly 10 may further be used to measure one or more properties of the blood (e.g., a “central venous oxygen saturation”), administer fluid or blood products for a large volume resuscitation, and/or measure a central venous pressure.

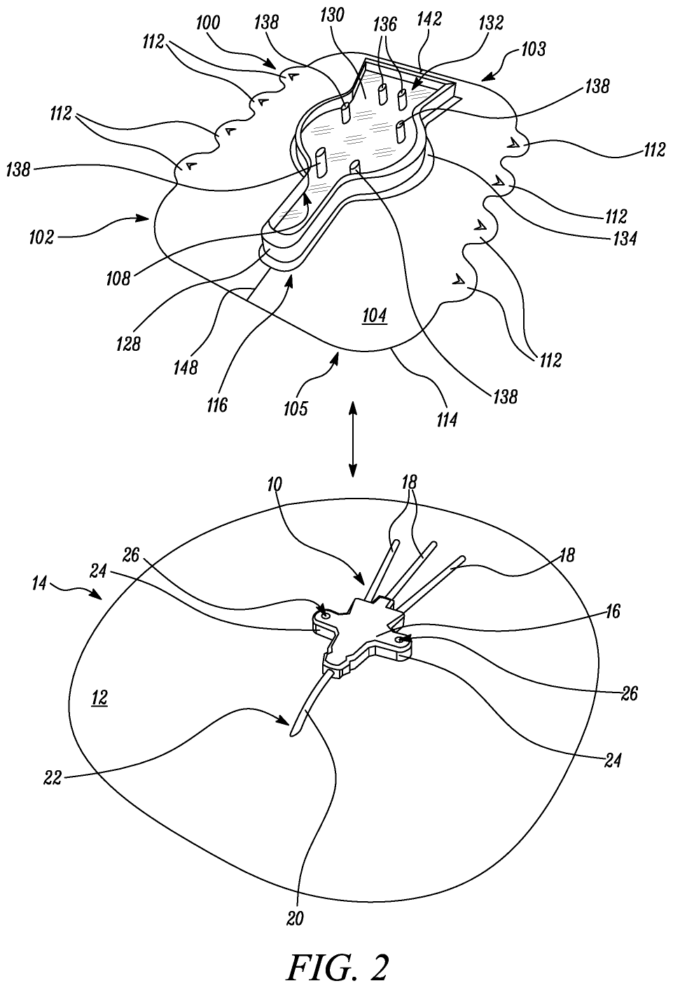

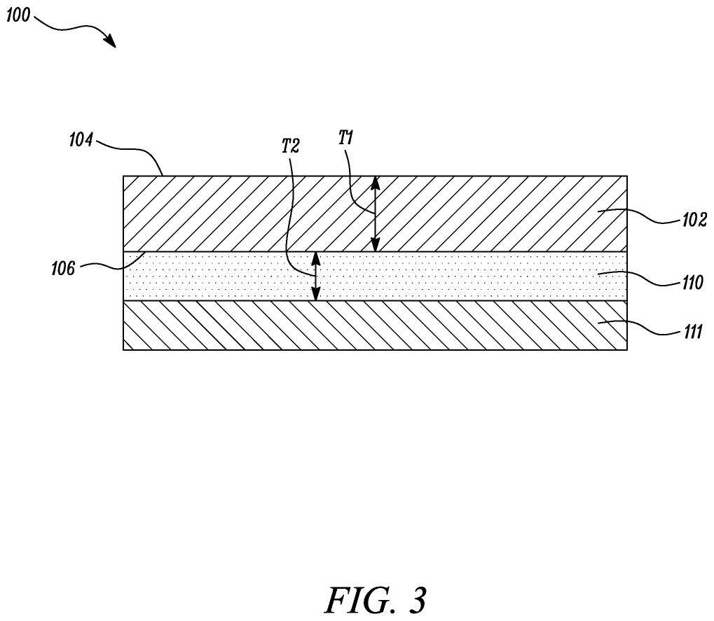

FIG. 2 illustrates a partially exploded perspective view of a securement device 100 for use with the catheter assembly 10. The securement device 100 includes a base 102 having a first major surface 104 and an opposing second major surface 106 (shown in FIG. 3). The second major surface 106 contacts the skin 12 of the user 14 proximate the injection site 22 whereas the first major surface 104 is disposed opposite to the second major surface 106. The base 102 is removably secured to the skin 12 of the user 14. In other words, the base 102 may be removably attached to and detached from the skin 12 of the user 14 when desired. In some embodiments, the base 102 may be removably secured to the skin 12 of the user 14 by a suitable adhesive, such as a pressure-sensitive adhesive.

The base 102 includes a stretchable polymeric material. The base 102 may be made of a stretchable material that may conform to undulated surfaces (e.g., the skin 12 of the user 14). Therefore, in some embodiments, the base 102 may be configured to exhibit high elasticity. In some embodiments, a modulus of elasticity of the base 102 may be at least about 2,500 pounds per square inch (psi) or at least about 3,000 psi. In some embodiments, the modulus of elasticity of the base 102 may be less than about 50,000 psi. In some embodiments, the modulus of elasticity of the base 102 may be between about 5,000 psi and about 30,000 psi. In some embodiments, the base 102 may have a high tensile strength, a low elastic recovery, and a high elongation at break.

In some embodiments, the base 102 includes at least one of a polymeric film, a polymeric foam, a polymeric hydrocolloid, and a polymeric alginate. In some embodiments, the base 102 may include polyolefins, such as polyethylene, including high density polyethylene, low density polyethylene, linear low density polyethylene, and linear ultra-low density polyethylene, polypropylene, and polybutylenes; vinyl copolymers, such as polyvinyl chlorides, both plasticized and unplasticized, and polyvinyl acetates; olefinic copolymers, such as ethylene/methacrylate copolymers, ethylene/vinyl acetate copolymers, acrylonitrile-butadiene-styrene copolymers, and ethylene propylene copolymers; acrylic polymers and copolymer; and combinations thereof. In some embodiments, the base 102 may be in a form of single or multi-layer films, non-woven films, porous films, foam-like films, and combinations thereof. In some embodiments, the base 102 may be fabricated using any suitable method of film forming, such as extrusion, co-extrusion, solvent casting, foaming, non-woven technology, and the like.

In some embodiments, the base 102 may be a passive dressing. In other words, in some embodiments, the base 102 may be non-occlusive. However, in some embodiments, the base 102 may be an interactive dressing. In other words, in some embodiments, the base 102 may be semi-occlusive or occlusive. Therefore, in some embodiments, the base 102 may act as a barrier against penetration of bacteria. In some cases, the base 102 may maintain hydration of an area of the skin 12 at which the base 102 is removably secured and may reduce infection of the area of the skin 12 at which the base 102 is removably secured.

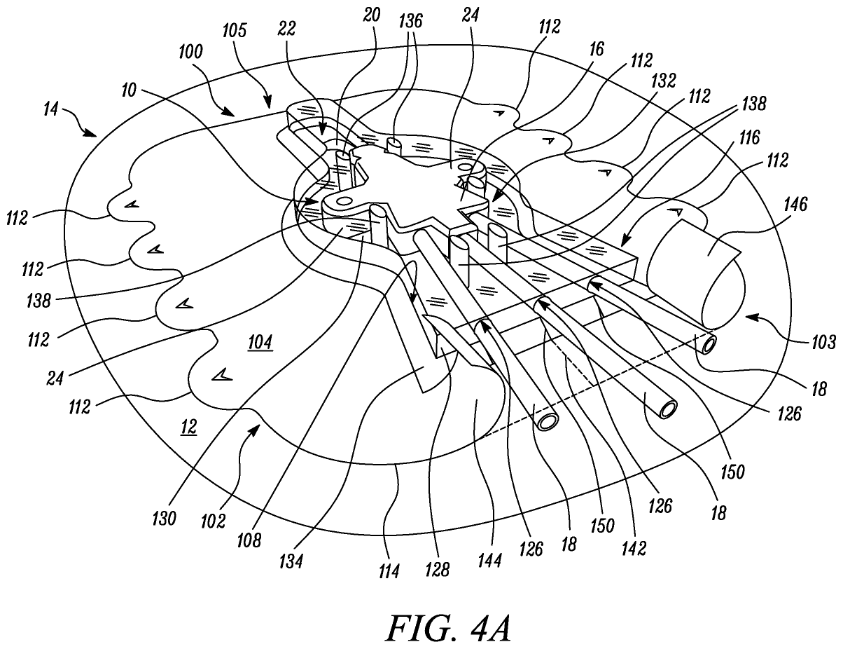

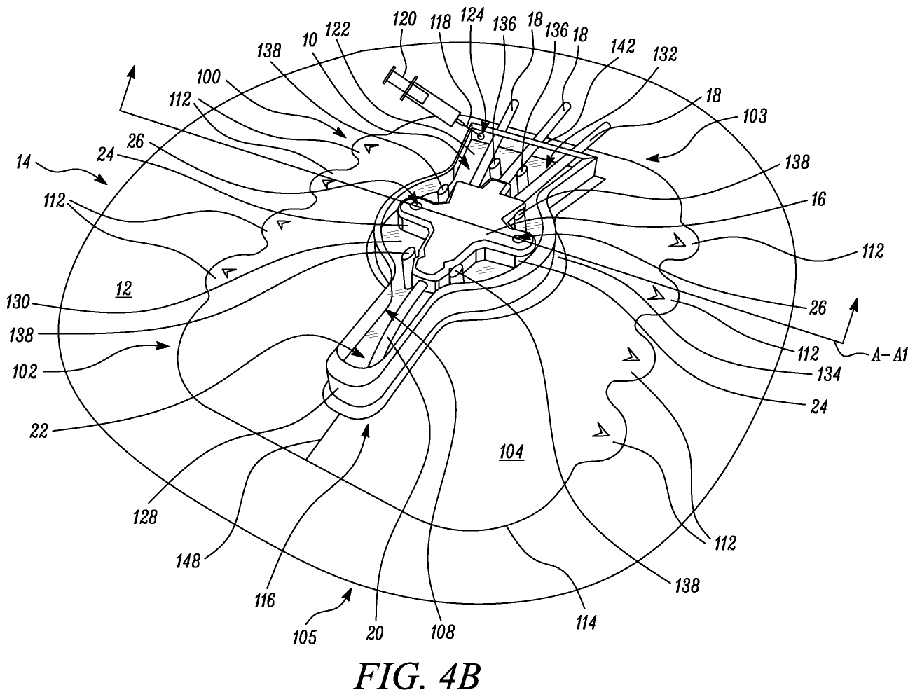

The base 102 defines a first end 103 and a second end 105 opposite the first end 103. The base 102 defines a first slit 142 (shown in FIGS. 2, 4A, and 4B) extending between a perimeter 114 of the base and a rim 134. Specifically, the first slit 142 allows passage of the lumens 18 for receipt of the lumens 18 within a casing 116 of the securement device 100. The first slit 142 is defined proximate the first end 103 of the base 102. Further, the base 102 defines a pair of straps 144, 146 (shown in FIG. 4A). The first slit 142 is defined such that the first slit 142 allows the pair of straps 144, 146 to open or close, as per application requirements. For example, the straps 144, 146 may be in an open position during receipt and/or removal of the lumens 18 from the casing 116. Moreover, the straps 144, 146 may be disposed in a closed position (as illustrated by hidden lines) once the lumens 18 are engaged with the casing 116. Further, the base 102 defines another slit 148 proximate the second end 105 of the base 102. The slit 148 extends between the perimeter 114 of the base 102 and the rim 134. In some embodiments, the base 102 may omit the slit 148.

The base 102 has a thickness T1 (shown in FIG. 3) defined between the first major surface 104 and the second major surface 106. The thickness T1 of the base 102 may depend upon desired application attributes. For example, the thickness T1 of the base 102 may depend upon a design and/or shape of the base 102 to ensure integrity and handleability of the base 102. In some cases, the thickness T1 being lower than about 10 micrometers may not be suitable for strong adhesives. Further, in some cases, the thickness T1 being greater than about 1000 micrometers may require greater stretching forces for removal of the base 102 from the skin 12 than desired. Therefore, in some embodiments, the thickness T1 of the base 102 may range from about 10 micrometers to about 1000 micrometers. The base 102 includes a substantially rectangular shaped structure. The shape of the base 102 is designed in such a way that the base 102 covers the injection site 22 of the cannula 20. It should be noted that the base 102 may include any other shape, without limiting the scope of the present disclosure.

Further, the base 102 defines the through cavity 108 extending from the first major surface 104 to the second major surface 106. The base 102 is configured to be detachably secured to the skin 12 of the user 14, such that the injection site 22 associated with the catheter assembly 10 is disposed within the through cavity 108. The through cavity 108 receives at least a portion of the catheter assembly 10 therein. Specifically, the through cavity 108 receives the cannula 20, the catheter hub 16, and a portion of the lumens 18. In some embodiments, a shape of the through cavity 108 confirms with a shape of the catheter assembly 10, so that the catheter assembly 10 can be received therein. Thus, the through cavity 108 may have any suitable shape to partially enclose the catheter assembly 10.

FIG. 3 illustrates a schematic cross-sectional view of the base 102. As shown in FIG. 3, the securement device 100 also includes an adhesive layer 110 at least partially disposed on the second major surface 106 of the base 102 and configured to detachably secure the base 102 to the skin 12 of the user 14. More particularly, the second major surface 106 of the base 102 is at least partially covered with the adhesive layer 110 to firmly secure the securement device 100 onto the skin 12 of the user 14. In the illustrated embodiment of FIG. 3, the adhesive layer 110 has a thickness T2. In some embodiments, the thickness T2 of the adhesive layer 110 may range from about 10 micrometers to about 400 micrometers. The adhesive layer 110 includes a stretch release adhesive. In some cases, an adhesive strength of the stretch-release adhesive may be less than a cohesive strength of the stretch-release adhesive, such that upon stretching of the base 102, adhesion capabilities of the stretch-release adhesive may essentially disappear. Specifically, a tack of the stretch-release adhesive may be lost upon stretching of the base 102. Therefore, in some embodiments, the base 102 may be easily removed from the skin 12 of the user 14 by stretching the base 102 based on gripping one or more pull tabs 112.

The securement device 100 further includes a release liner 111. The release liner 111 may protect the adhesive layer 110 from contaminants, such as dust, debris, and the like, prior to use of the securement device 100. The release liner 111 may be manufactured using a plastic base material. The release liner 111 includes a substantial rectangular shape similar to the shape of the base 102. The release liner 111 may be peeled off from the adhesive layer 110 prior to removable securement of the base 102 to the skin 12 of the user 14. The release liner 111 may include any suitable material, such as Polyethylene Terephthalate (PET), High-Density Polyethylene (HDPE), Polyvinyl Chloride (PVC), Polypropylene (PP), and the like. In the illustrated embodiment of FIG. 3, the release liner 111 does not cover the pull tabs 112. Alternatively, in other embodiments, the release liner 111 may cover the pull tabs 112 as well as the adhesive layer 110.

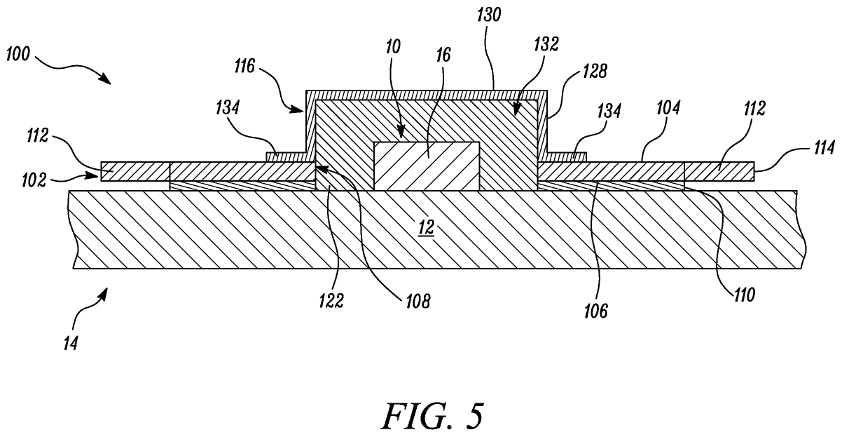

As shown in FIGS. 4A, 4B, and 5, the base 102 further includes the one or more pull tabs 112 disposed around the perimeter 114 of the base 102 and free of the adhesive layer 110 (see FIG. 5). The pull tabs 112 are adhesive free zones, such that the pull tabs 112 are not attached to the skin 12 of the user 14. The one or more pull tabs 112 are configured to be gripped to detach the base 102 from the skin 12. Specifically, the pull tabs 112 allow gripping of the base 102 for application and removal of the securement device 100 and also allow a clinician or any other medical professional to peel-off the release liner 111 (not shown) for application of the securement device 100 onto the skin 12 of the user 14. In other words, in the illustrated embodiment of FIGS. 4A, 4B, and 5, the pull tabs 112 are not adhered to the skin 12 of the user 14. Thus, the pull tabs 112 may be gripped by clinicians or other medical professionals. Further, in some embodiments, for removal of the base 102 from the skin 12 of the user 14, the base 102 may be stretched by gripping and pulling the pull tabs 112.

FIG. 4A illustrates a schematic perspective view of the securement device 100. In the illustrated embodiment of FIG. 4A, the one or more pull tabs 112 includes multiple pairs of opposing pull tabs 112. Thus, for removal of the base 102 from the skin 12 of the user 14, the base 102 may be stretched by gripping the pairs of opposing pull tabs 112. The pairs of opposing pull tabs 112 may facilitate removal of the base 102 from the skin 12 of the user 14. In the illustrated embodiment of FIG. 4A, the base 102 includes four pairs of opposing pull tabs 112, i.e., eight pull tabs 112 in total. The pull tabs 112 defines a substantially semi-circular shape. In other embodiments, the pull tabs 112 may include any shape and number of the pull tabs 112, without any limitations. In some examples, the pull tabs 112 may include markings thereon that indicate a direction along which the pull tabs 112 may be pulled for removal of the securement device 100.

Further, the securement device 100 includes the casing 116 mounted on the first major surface 104 of the base 102 and enclosing the through cavity 108 and the catheter assembly 10. The casing 116 is configured to engage with the catheter assembly 10 to secure the catheter assembly 10 within the through cavity 108. The casing 116 defines a wall 128 and an upper covering 130. The wall 128 extends generally perpendicular to the upper covering 130 and the base 102. The wall 128 and the upper covering 130 together define a volume 132 of the casing 116, such that the catheter assembly 10 is partially receivable within the volume 132.

Further, the casing 116 includes the rim 134 attached to the first major surface 104 of the base 102. Specifically, the rim 134 allows attachment of the casing 116 to the base 102. The rim 134 extends from the wall 128 of the casing 116 and attaches to the base 102. In some embodiments, the rim 134 may include an adhesive that allows attachment of the rim 134 to the first major surface 104. Further, the rim 134 includes at least one second slit 150, such that the first slit 142 (see FIGS. 2 and 4A) and the at least one second slit 150 allow receipt of the at least one lumen 18 within the casing 116. The second slit 150 is disposed proximate the first end 103 of the base 102. In the illustrated embodiment of FIG. 4A, the rim 134 includes three second slits 150 (two of which are illustrated in FIG. 4A) that are equidistantly spaced apart from each other. Each second slit 150 allows passage of a corresponding lumen 18 therethrough for receipt of the corresponding lumen 18 within the casing 116. It should be noted that the rim 134 may include any number of second slits 150 based on a total number of the lumens 18.

Further, each second slit 150 aligns with a corresponding self-healing slit (not shown) in the wall 128 of the casing 116 for passage of the lumen 18 therethrough. Specifically, for receipt of the lumens 18 within the casing 116, the first slit 142 allows the straps 144, 146 to separate from each other to dispose the straps 144, 146 in the open position. Further, each lumen 18 is pushed through a corresponding second slit 150 and a corresponding self-healing slit until the lumen 18 is received within a self-healing hole 126. Once the lumens 18 are received within corresponding self-healing holes 126, the straps 144, 146 may be closed to secure the straps 144, 146 to the skin 12 of the user 14.

The casing 116 includes a material having the self-healing property. The material of the casing 116 includes at least one of a shape memory polymeric material and a thermoformed plastic material. For example, the material of the casing 116 may include polyvinyl chloride, polycarbonate, and the like. The shape memory polymeric material has an ability to return from a deformed/ruptured state to its original shape. Further, the material also tends to move to a ruptured state from its original shape upon insertion of a foreign object at least partially inside the material. If the foreign object is removed, the material returns to its original shape. If the foreign object remains inserted, the material forms a seal around the foreign object. The thermoformed plastic material has stretching properties at specific temperatures. The material of the casing 116 allows a needle 118 or the lumens 18 to rupture the casing 116. Specifically, as the material of the casing 116 includes self-healing properties, ruptured areas of the casing 116 return to their original shapes and sizes as soon as the needle 118 and/or the lumens 18 are removed, or the area proximate the rupture heals itself to prevent entry of any foreign elements into the casing 116.

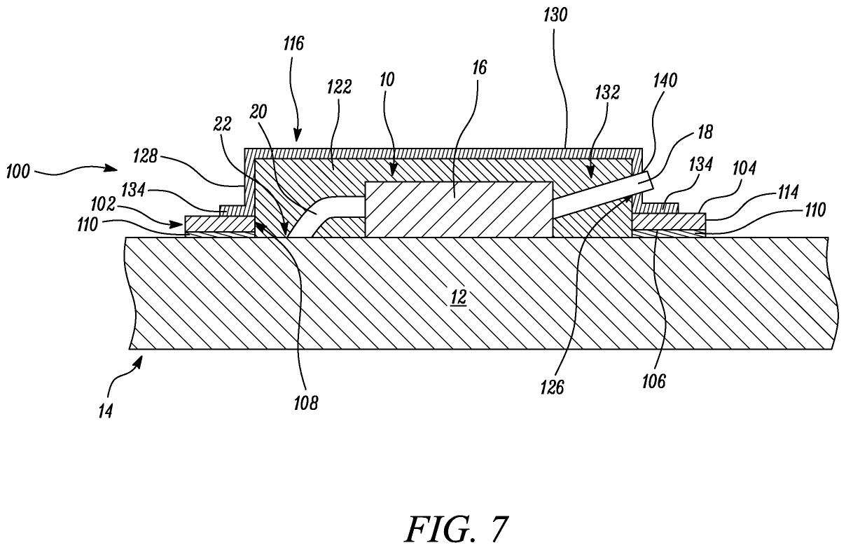

The casing 116 at least partially ruptures to receive the at least one lumen 18 of the catheter assembly 10 therethrough. Further, upon receiving the at least one lumen 18 of the catheter assembly 10 therethrough, the casing 116 forms a seal 140 (shown in FIG. 7) around the at least one lumen 18. In some embodiments, the casing 116 defines the one or more self-healing holes 126 to receive the lumens 18 therethrough. A total number of the self-healing holes 126 may correspond to the total number of the lumens 18. In the illustrated embodiment of FIG. 4A, the casing 116 includes three self-healing holes 126. Further, the casing 116 includes the one or more self-healing slits. Specifically, the casing 116 includes three self-healing slits corresponding to the number of self-healing holes 126. Each self-healing slit communicates with a corresponding self-healing hole 126. The self-healing slits and the self-healing holes 126 are formed within the wall 128 of the casing 116.

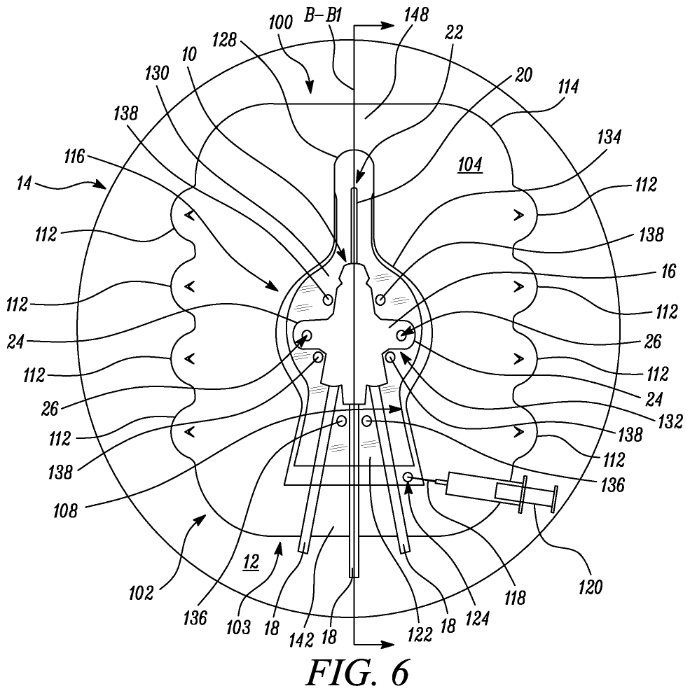

Referring to FIGS. 4B and 6, upon insertion of the needle 118 of a syringe 120 for injection of an anti-microbial gel 122 within the casing 116, the casing 116 forms a self-healing aperture 124 to receive the needle 118 therethrough. Moreover, once the syringe 120 is removed, the self-healing aperture 124 so formed seals owing to the self-healing material of the casing 116. Specifically, the casing 116 forms a seal around the needle 118. It should be noted that a location of the self-healing aperture 124 illustrated herein is exemplary in nature, and the self-healing aperture 124 may be located at any other place of the casing. Further, the material of the casing 116 is transparent or translucent. The transparent or translucent material of the casing 116 allows clinicians or other medical professionals to observe the catheter assembly 10 and the injection site 22 when the securement device 100 is secured to the skin 12.

The casing 116 may have any suitable shape to enclose the catheter assembly 10 at least partially. In other words, the shape of the casing 116 may depend upon the catheter assembly 10. Further, the shape of the casing 116 is substantially similar to the shape of the through cavity 108. Moreover, a perimeter area of the casing 116 may be slightly greater than or substantially equal to a perimeter area of the through cavity 108 such that the casing 116 encloses the through cavity 108.

Referring to FIGS. 4B, 6, and 7, the casing 116 also includes a plurality of guide pins 136 (as shown in FIGS. 2, 4B, and 6) engaging with the at least one lumen 18. The guide pins 136 engage with the one or more lumens 18 to restrict the movement of the one or more lumens 18 within the casing 116. The guide pins 136 project vertically downwards from the upper covering 130 of the casing 116 such that the guide pins 136 are substantially parallel to the wall 128 of the casing 116. In the illustrated embodiment of FIGS. 4B, 6, and 7, the casing 116 includes two guide pins 136. The guide pins 136 include an elliptical cross-section. In other embodiments, the casing 116 may include any number of guide pins 136 and any other shape, without any limitations. For example, the guide pins 136 may have a circular cross-section, a square cross-section, a rectangular cross-section, and the like. Further, a height of the guide pins 136 may be substantially equal to a height of the wall 128.

The casing 116 further includes a plurality of stabilization lugs 138 (as shown in FIGS. 2, 4B, and 6) engaging with the catheter assembly 10 to secure the catheter assembly 10 within the through cavity 108. More particularly, the stabilization lugs 138 engage with the catheter hub 16 to restrict the movement of the catheter hub 16 relative to the casing 116. The stabilization lugs 138 project vertically downwards from the upper covering 130 of the casing 116 such that the stabilization lugs 138 are substantially parallel to the wall 128 and the guide pins 136. In the illustrated embodiment of FIGS. 4B, 6, and 7, the casing 116 includes four stabilization lugs 138. The stabilization lugs 138 include an elliptical cross-section. In the illustrated embodiment of FIGS. 4B, 6, and 7, the stabilization lugs 138 are disposed in a manner such that the plurality of stabilization lugs 138 together accommodate the catheter hub 16 therein. In some embodiments, the stabilization lugs 138 may align with the holes 26 in the catheter hub 16 for receipt of the stabilization lugs 138 within the holes 26 during securement of the securement device 100 on the skin 12 of the user 14. In such embodiments, the stabilization lugs 138 may have a shape that allows receipt of the stabilization lugs 138 within the holes 26. For example, the stabilization lugs 138 may have a circular shape.

In other embodiments, the casing 116 may include any number of stabilization lugs 138 and any other shape, without any limitations. For example, the stabilization lugs 138 may have a circular cross-section, a square cross-section, a rectangular cross-section, and the like. Further, a height of the stabilization lugs 138 may be substantially equal to the height of the wall 128 of the casing 116 and the guide pins 136. It should be noted that the shape and size of the stabilization lugs 138 and the guide pins 136 may be identical or non-identical, without limiting the scope of the present disclosure. The guide pins 136 and the stabilization lugs 138 may together eliminate any undesirable movement of the catheter assembly 10 relative to the casing 116.

Further, the anti-microbial gel 122 is received within the casing 116 and at least partially fills the through cavity 108. For example, the anti-microbial gel 122 may form a layer on the skin 12 of the user 14. Further, the anti-microbial gel 122 may also partially or completely fill the volume 132 of the casing 116. The anti-microbial gel 122 is injected within the casing 116 after the securement device 100 is placed at the injection site 22. The anti-microbial gel 122 can be injected within the casing 116 using the syringe 120 (see FIGS. 4B and 6). Further, upon insertion of the needle 118 (see FIGS. 4B and 6) of the syringe 120 for injection of the anti-microbial gel 122 within the casing 116, the casing 116 forms the self-healing aperture 124 (see FIGS. 4B and 6) to receive the needle 118 therethrough. The anti-microbial gel 122 may contain anti-microbial agents. The anti-microbial gel 122 may include at least one of silver nanoparticles, gold nanoparticles, and antibiotics. The anti-microbial gel 122 may include a high viscosity fluid which surrounds and protects the injection site 22 from infections and keeps the injection site 22 sterile. Further, the anti-microbial gel 122 may nourish the skin 12 and may keep the skin 12 moist.

Therefore, the securement device 100 may secure the catheter assembly 10 firmly onto the skin 12 of the user 14. Furthermore, the securement device 100 may inhibit movement of the catheter assembly 10. Consequently, the securement device 100 may prevent various complications that may occur due to micro-movement of the catheter assembly 10. The securement device 100 may further prevent infection at the injection site 22. Moreover, the securement device 100 may provide improved comfort to the user 14 during use.

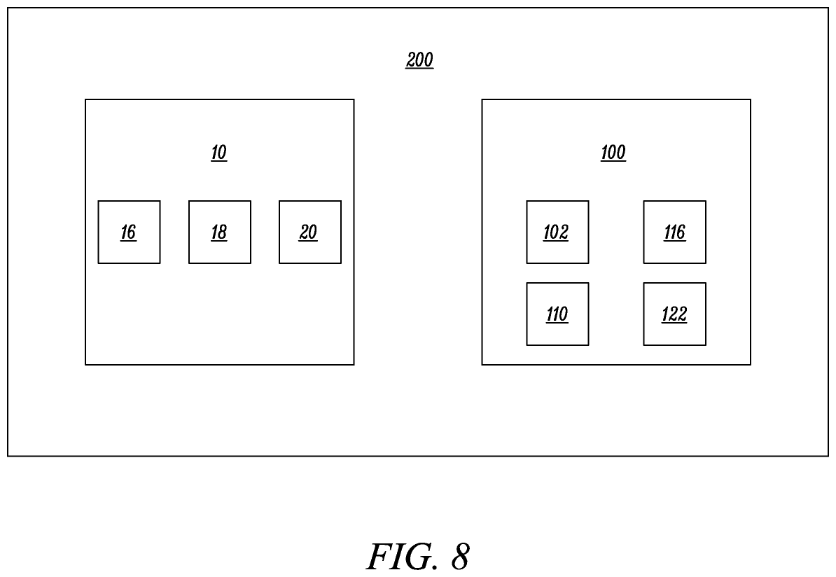

FIG. 8 illustrates a schematic block diagram of a kit 200 according to an embodiment of the present disclosure.

Referring to FIGS. 4A, 4B, 6, and 8, the kit 200 includes the catheter assembly 10. In the illustrated embodiment of FIGS. 4A, 4B, 6, and 8, the catheter assembly 10 includes the catheter hub 16, the one or more lumens 18 at least partially received within the catheter hub 16, and the cannula 20 at least partially received within the catheter hub 16.

The kit 200 further includes the securement device 100. The securement device 100 is used for securing the catheter assembly 10 to the skin 12 of the user 14. The securement device 100 includes the base 102 and the casing 116. In some embodiments, the securement device 100 includes the adhesive layer 110. In some embodiments, the kit 200 may include the syringe 120 and the needle 118. In some embodiments, the kit 200 includes the anti-microbial gel 122 received within the casing 116 and at least partially filling the through cavity 108. The anti-microbial gel 122 may be in a form of liquid or a gel. Upon insertion of the needle 118 of the syringe 120 for injection of the anti-microbial gel 122 within the casing 116, the casing 116 forms the self-healing aperture 124 to receive the needle 118 therethrough.

In some embodiments, the kit 200 may further include gloves (not shown). In some embodiments, the kit 200 may further include a sterilizing material (not shown). In some embodiments, the kit 200 may further include a cloth or other absorbent material (not shown). In some embodiments, the kit 200 may further include cleaning articles (not shown), such as cleaning cloth, cotton balls, cotton swabs, and the like.

The kit 200 may be available to clinicians (or medical professionals) for use in a sterilized package. The clinician may remove the release liner 111 (see FIG. 3) and detachably attach the securement device 100 onto the skin 12. The catheter assembly 10 may therefore be secured and stabilized. In some cases, the clinician may further apply a window dressing over the injection site 22 to provide extra protection from infection at the injection site 22. The through cavity 108 may allow the securement device 100 to be applied to a cannula (e.g., the cannula 20) already inserted into an injection site (e.g., the injection site 22 in FIG. 1).

The material of the casing 116 includes at least one of the shape memory polymeric material and the thermoformed plastic material. The casing 116 of the securement device 100 may allow the catheter assembly 10 to be inspected whenever desired. For example, the casing 116 may be made of a transparent material to enable inspection of the catheter assembly 10. Further, the securement device 100 may allow the clinician to change a position of the securement device 100 without removal of the catheter assembly 10 from the skin 12 of the user 14.

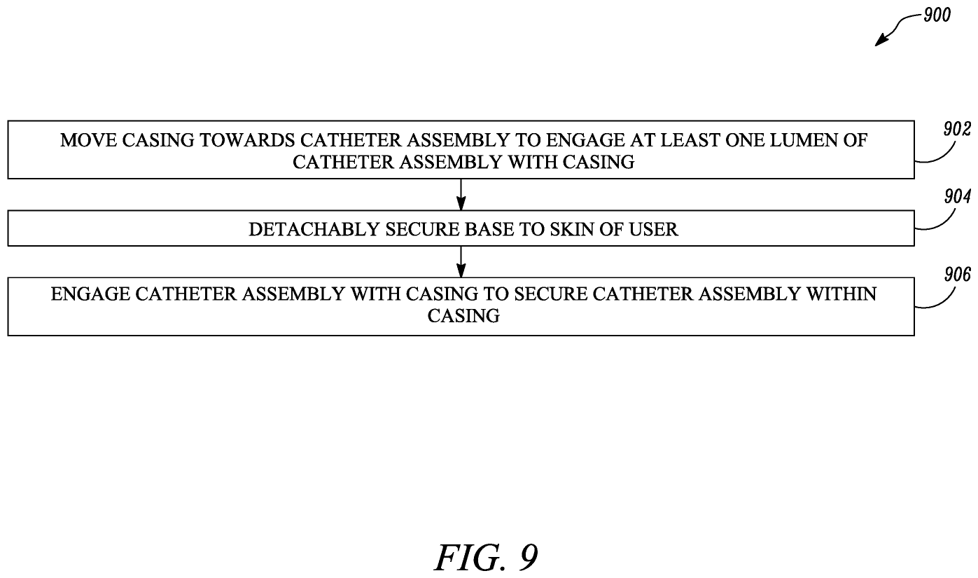

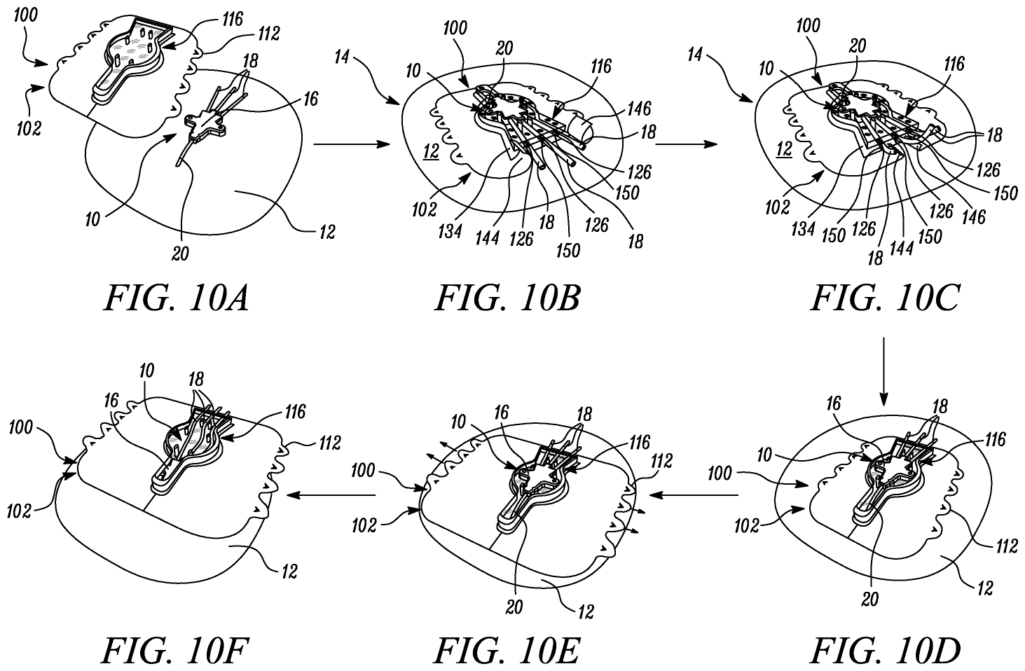

FIG. 9 illustrates a flowchart of a method 900 of using the securement device 100 including the base 102 and the casing 116 mounted on the first major surface 104 of the base 102. FIGS. 10A-10F illustrate exemplary steps of securing and removal of the securement device 100 from the skin 12 of the user 14. The method 900 will further be described with reference to FIGS. 9, and 10A-10F.

FIG. 10A shows an exploded view of the securement device 100 and the catheter assembly 10. The securement device 100 may be an integral part of the catheter assembly 10, or it can be retrofitted on the catheter assembly 10. The skin 12 of the user 14 is cleaned and sterilized before injecting the cannula 20 of the catheter assembly 10. Further, the catheter hub 16 of the catheter assembly 10 is disposed on the skin 12 of the user 14. The method 900 includes a step wherein the at least one lumen 18 is at least partially received within the catheter hub 16. Subsequently, the cannula 20 of the catheter assembly 10 is inserted into the injection site 22 located on the skin 12 of the user 14. The cannula 20 is at least partially received within the catheter hub 16.

At step 902, the base 102 is detachably secured to the skin 12 of the user 14, such that the second major surface 106 of the base 102 opposite to the first major surface 104 faces the skin 12. Further, the base 102 is secured to the skin 12 of the user 14 by the adhesive layer 110 at least partially disposed on the second major surface 106 of the skin 12. The release liner 111 disposed on the adhesive layer 110 is removed and the base 102 of the securement device 100 is placed on the injection site 22. As illustrated in FIG. 10B, the straps 144, 146 are in the open position when the base 102 is being secured to the skin 12 of the user 14.

At step 904, the casing 116 is moved towards the catheter assembly 10 to engage the at least one lumen 18 of the catheter assembly 10 with the casing 116, such that the casing 116 at least partially ruptures and receives the at least one lumen 18 therethrough (as shown in FIG. 10B). The casing 116 includes the self-healing material, such that upon receiving the at least one lumen 18 therethrough, the casing 116 forms the seal 140 around the at least one lumen 18. FIG. 10B illustrates receipt of the three lumens 18 within the casing 116. As illustrated in FIG. 10B, the step of engaging the at least one lumen 18 of the catheter assembly 10 with the casing 116 includes passing the at least one lumen 18 through the first slit 142 (see FIGS. 2 and 4B) in the base 102 and the at least one second slit 150 in the rim 134 of the casing 116. More particularly, the first slit 142 allows the straps 144, 146 to be disposed in the open position so that each lumen 18 can pass through a corresponding second slit 150 for receipt of the lumen 18 within a corresponding self-healing hole 126. As shown in FIG. 10C, once the lumens 18 engage with the casing 116, a portion of the lumens 18 that is disposed outside of the casing 116 is held in a bent position so that the straps 144, 146 can be moved to the closed position for completely securing the base 102 on the skin 12 of the user 14. On closure of the straps 144, 146, the lumens 18 may return to their original position.

At step 906, the catheter assembly 10 engages with the casing 116 to secure the catheter assembly 10 within the casing 116 (as shown in FIG. 10D). The catheter assembly 10 is at least partially received within the through cavity 108 of the base 102 and enclosed by the casing 116. Further, the at least one lumen 18 is engaged with the one or more guide pins 136 of the casing 116. Moreover, engaging of the catheter assembly 10 with the casing 116 further includes engaging the catheter assembly 10 with the one or more stabilization lugs 138 of the casing 116. Once the base 102 is placed on the injection site 22, the first major surface 104 of the base 102 may be pressed lightly to firmly secure the base 102 on the skin 12 of the user 14.

FIG. 10D illustrates receipt of the catheter hub 16, some portion of corresponding lumens 18, and the cannula 20 within the through cavity 108. Further, the guide pins 136 are shown engaged with the lumens 18 to restrict the movement of the lumens 18 within the casing 116. Moreover, the stabilization lugs 138 are shown engaged with the catheter hub 16 to restrict the movement of the catheter hub 16 within the casing 116.

Further, the method 900 also includes inserting the syringe 120 of the needle 118 through the casing 116, such that the casing 116 forms the self-healing aperture 124 around the needle 118. Moreover, the anti-microbial gel 122 is injected within the casing 116 via the syringe 120, such that the anti-microbial gel 122 at least partially fills the through cavity 108. The anti-microbial gel 122 surrounds and protects the injection site 22 from one or more microbial infections and keep the injection site 22 sterile.

The method 900 further includes gripping one or more pull tabs 112 disposed at the perimeter 114 of the base 102. The one or more pull tabs 112 are free of the adhesive layer 110. Moreover, the base 102 is stretched via the one or more pull tabs 112 to release the base 102 from the skin 12 of the user 14. Referring to FIG. 10E the pull tabs 112 can be easily gripped by the user 14 as the pull tabs 112 do not contain the adhesive layer 110. Further, stretching the base 102 deforms the adhesive layer 110 that leads to lose of adhesion property of the adhesive layer 110, therefore the base 102 can be released easily from the skin 12 of the user 14. The base 102 is illustrated in a stretched condition in FIGS. 10E and 10F due to the pulling of the base 102 based on application of a pull force on the pull tabs 112.

The method 900 also includes moving the casing 116 away from the catheter assembly 10, such that the casing 116 at least partially ruptures and disengages from the at least one lumen 18. FIG. 10F illustrates a step of removal of the lumens 18 from the casing 116. Specifically, as the casing 116 is moved away from the catheter assembly 10, the catheter hub 16 disengages form the casing 116 and the casing 116 at least partially ruptures and disengages from the lumens 18. Further movement of the casing 116 causes the lumens 18 to completely disengage from the casing 116, thereby releasing the securement device 100 from the skin 12 of the user 14. In some embodiments, the step of removal of the lumens 18 may include passage of the lumen 18 through a corresponding self-healing slit and a corresponding second slit 150 for disengagement of the lumens 18 from the casing 116. Further, the straps 144, 146 may be disposed in the open position to facilitate complete removal of the lumens 18 form the securement device 100.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

1. A securement device for securing a catheter assembly, the securement device comprising:
a base having a first major surface and an opposing second major surface, the base defining a through cavity extending from the first major surface to the second major surface, wherein the base is configured to be detachably secured to a skin of a user, such that an injection site associated with the catheter assembly is disposed within the through cavity, and wherein the through cavity receives at least a portion of the catheter assembly therein; and
a casing mounted on the first major surface of the base and enclosing the through cavity and the catheter assembly, wherein the casing is configured to engage with the catheter assembly to secure the catheter assembly within the through cavity, and wherein the casing comprises a material having a self-healing property, such that:
the casing at least partially ruptures to receive at least one lumen of the catheter assembly therethrough; and
upon receiving the at least one lumen of the catheter assembly therethrough, the casing forms a seal around the at least one lumen.

2. The securement device of claim 1, further comprising an adhesive layer at least partially disposed on the second major surface of the base and configured to detachably secure the base to the skin of the user.

3. The securement device of claim 2, wherein the base further comprises one or more pull tabs disposed at a perimeter of the base and free of the adhesive layer, and wherein the one or more pull tabs are configured to be gripped to detach the base from the skin.

4. The securement device of claim 2, wherein the adhesive layer comprises a stretch release adhesive.

5. The securement device of claim 1, wherein the base further comprises a stretchable polymeric material.

6. The securement device of claim 1, wherein the material of the casing is transparent or translucent.

7. The securement device of claim 1, wherein the material of the casing comprises at least one of a shape memory polymeric material and a thermoformed plastic material.

8. The securement device of claim 1, wherein the casing further comprises a rim attached to the first major surface of the base.

9. The securement device of claim 8, wherein the base further comprises a first slit extending between a perimeter of the base and the rim.

10. The securement device of claim 9, wherein the rim comprises at least one second slit, such that the first slit and the at least one second slit allow receipt of the at least one lumen within the casing.

11. The securement device of claim 1, wherein, upon insertion of a needle of a syringe for injection of an anti-microbial gel within the casing, the casing forms a self-healing aperture to receive the needle therethrough.

12-13. (canceled)

14. A kit comprising:
a catheter assembly comprising a catheter hub, at least one lumen at least partially received within the catheter hub, and a cannula at least partially received within the catheter hub, wherein the cannula is configured to be at least partially inserted into a skin of a user at an injection site; and
a securement device comprising:
a base having a first major surface and an opposing second major surface, the base defining a through cavity extending from the first major surface to the second major surface, wherein the base is configured to be detachably secured to the skin of the user, such that an injection site is disposed within the through cavity, wherein the through cavity receives the catheter hub therein, and wherein the through cavity at least partially receives the at least one lumen and the cannula therein; and
a casing mounted on the first major surface of the base and enclosing the through cavity and the catheter assembly, wherein the casing is configured engage with the catheter assembly to secure the catheter assembly within the through cavity, and wherein the casing comprises a material having a self-healing property, such that:
the casing at least partially ruptures to receive the at least one lumen of the catheter assembly therethrough; and
upon receiving the at least one lumen of the catheter assembly therethrough, the casing forms a seal around the at least one lumen.

15. The kit of claim 14, further comprising an adhesive layer at least partially disposed on the second major surface of the base and configured to detachably secure the base to the skin of the user.

16-28. (canceled)

29. A method of using a securement device comprising a base and a casing mounted on a first major surface of the base, the method comprising:
detachably securing the base to a skin of a user, such that a second major surface of the base opposite to the first major surface faces the skin, and the catheter assembly is at least partially received within a through cavity of the base and enclosed by the casing;
engaging at least one lumen of the catheter assembly with the casing, such that the casing at least partially ruptures and receives the at least one lumen therethrough, wherein the casing comprises a self-healing material, such that upon receiving the at least one lumen therethrough, the casing forms a seal around the at least one lumen; and
engaging the catheter assembly with the casing to secure the catheter assembly within the casing.

30. The method of claim 29, wherein the base is secured to the skin of the user by an adhesive layer at least partially disposed on the second major surface of the skin.

31. The method of claim 30, further comprising:
gripping one or more pull tabs disposed at a perimeter of the base, wherein the one or more pull tabs are free of the adhesive layer; and
stretching, via the one or more pull tabs, the base to release the base from the skin of the user.

32. The method of claim 29, wherein engaging the at least one lumen of the catheter
assembly with the casing further comprises passing the at least one lumen through a first slit in the base and at least one second slit in a rim of the casing.

33. The method of claim 29, further comprising:
disposing a catheter hub of the catheter assembly on the skin of the user, wherein the at least one lumen is at least partially received within the catheter hub; and
inserting a cannula of the catheter assembly into an injection site located on a skin of the user, wherein the cannula is at least partially received within the catheter hub.

34. The method of claim 29, further comprising:
inserting a needle of a syringe through the casing, such that the casing forms a self-healing aperture around the needle; and
injecting, via the syringe, an anti-microbial gel within the casing, such that the anti-microbial gel at least partially fills the through cavity.

35. The method of claim 29, further comprising engaging the at least one lumen with one or more guide pins of the casing.

36-37. (canceled)
